(19) United States
(12) Patent Application Publication
(10) Pub. No.: US 2006/0123492 A1
(43) Pub. Date: Jun. 8, 2006

(54) Methods of identifying modulators of NMUR2-mediated activity

(76) Inventors: Stanley J. Wiegand, Croton-on-Hudson, NY (US); Andrew J. Murphy, Croton-on-Hudson, NY (US); Susan Croll-Kalish, Tarrytown, NY (US); Richard Torres, New York, NY (US)

Correspondence Address: REGENERON PHARMACEUTICALS, INC, 777 OLD SAW MILL RIVER ROAD, TARRYTOWN, NY, 10591, US

(21) Appl. No.: 11/332,753

(22) Filed: Jan. 13, 2006

Related U.S. Application Data
(62) Division of application No. 10/758,798, filed on Jan. 16, 2004.
(60) Provisional application No. 60/443,782, filed on Jan. 30, 2003.
(60) Provisional application No. 60/483,994, filed on Jul. 1, 2003.

(51) Int. Cl. A01K 67/00; G01N 33/53; A61K 39/395

(52) U.S. Cl. 800/8; 435/7.1; 424/143.1

(57) ABSTRACT

Methods for screening for agents capable of modulating human neuromedin U receptor 2 (NMUR2) activity are provided, as well as therapeutic methods for treating NMUR2-mediated conditions. More specifically, methods are provided for identifying agents capable of inhibiting NMUR2-mediated pain or nociception.

### CROSS-REFERENCE TO RELATED APPLICATIONS

This application claims the benefit under 35 USC § 119(e) of U.S. Provisional 60/443,782 filed 30 Jan. 2003 and 60/483,994 filed 1 Jul. 2003, which applications are herein specifically incorporated by reference in their entirety.

### BACKGROUND OF THE INVENTION

1. Field of the Invention

This invention is related to methods of using G-protein-coupled receptor (GPCR) nucleic acids and polypeptides, more specifically, human neuromedin U receptor 2 (NMUR2) GPCR nucleic acids and polypeptides, as well as methods of making said polypeptides.

2. Description of Related Art

GPCR-66 and GPCR-66-like (NUMR2) are two structurally related GPCRs that have been identified as receptors for the neuropeptide, neuromedin U (NMU). This peptide is found in highest levels in the gut and in the genitourinary systems, but is also expressed in the spinal cord and in parts of the brain. (Raddatz el al. (2000) J. Biol. Chem. 275:32425-32459; Shan et al. (2000) J. Biol. Chem. 275:39482-39486; U.S. Pat. No. 6,461,836). It has been postulated that NMU is involved in the stimulation of smooth muscle, increase of blood pressure, regulation of adrenocortical function, and in the control of feeding, among others (Howard et al. (2000) Nature 406:71-74).

### BRIEF SUMMARY OF THE INVENTION

Although the genes encoding NMUR2 and its ligand are known, the function of NMUR2 in the modulation of nociception, pain and/or thermal sensation is now elucidated for the first time. The knowledge of the function of NMUR2 allows, for the first time, the development of screening and therapeutic methods leading to the development of a class of potent non-opiate analgesics unencumbered by one or more of the undesirable side effects of opiates or opiate-receptor ligands.

NMUR2 is expressed in neurons involved in the modulation of the sensations of nociception, pain and temperature, in particular in the relay of such information from the periphery to higher levels of the central nervous system. Accordingly, in a first aspect, the invention provides methods for screening for agents capable of binding a human NMUR2 protein or protein fragment having NMUR2 activity. More specifically, the invention provides methods of identifying agents capable of modulating (e.g., enhancing or inhibiting). The screening methods of the invention include in vitro and in vivo assays. Agents capable of modulating NMUR2-mediated activity preferably include agents capable of inhibiting NMUR2 modulation of pain sensation.

In one embodiment of an in vitro screening method of the invention, agents capable of binding the NMUR2 protein or protein fragment are identified in a cell-based assay system. More specifically, cells expressing a NMUR2 protein or a protein fragment having NMUR2 activity, are contacted with a test compound or a control compound, and the ability of the candidate compound to bind NMUR2 or a fragment thereof is determined. In a more specific competitive binding embodiment, the test compound is contacted with the cell in the presence of a NMUR2 ligand, and the ability of the test compound to bind NMUR2 in the presence of the competitive NMUR2 ligand is determined. In an even more specific embodiment, the NMUR2 ligand is labeled. Labeling of the NMUR2 ligand is by any method known to the art, including for example, radioactivity or fluorescence. In an even more specific embodiment, the NMUR2 ligand is neuromedin U (NMU), including naturally-occurring, recombinant, or derivatives of NMU.

In another embodiment, agents capable of binding a NMUR2 protein or protein fragment are identified in a cell-free assay system. More specifically, a native or recombinant human NMUR2 protein or protein fragment is contacted with a candidate compound or a control compound, and the ability of the candidate compound to bind NMUR2 or a fragment thereof is determined.

In another embodiment, agents capable of binding NMUR2 or a fragment thereof are identified in vivo in an animal system. More specifically, a candidate agent or a control compound is administered to a suitable animal, and the effect on NMUR2 modulation of pain is determined. Any suitable assay known to the art for determination of pain may be used, including reaction to heat or a tail flick assay.

In a second related aspect, the invention provides methods for identifying agents capable of inhibiting the activity of human NMUR2. More specifically, the invention provides methods of identifying agents which inhibit NMUR2 modulation of pain or nociception. In one embodiment, the agent capable of inhibiting NMUR2 is an antagonist to a natural NMUR2 ligand capable of binding to human NMUR2. In a more specific embodiment the antagonist is an antibody.

In a third aspect, the invention features a method of treating a NMUR2-mediated condition, comprising administering an agent capable of inhibiting NMUR2 activity. In one embodiment, the NMUR2-mediated condition is a chronic pain disease, such as chronic fatigue syndrome or fibromyalgia. In another embodiment, the NMUR2-mediated condition results from an injury to the body, including surgery, medical treatment, or accident. In one embodiment, the agent administered is a compound identified through a screening method of the invention.

In a related fourth aspect, the invention features a therapeutic method for inhibiting pain, comprising administering a therapeutically effective amount of an agent capable of effecting NMUR2 modulation of pain or nociception. In one embodiment, the agent is identified by the screening assay of the invention. In one embodiment, the agent is an inhibitor, such as an antagonist of NMUR2. In a more specific embodiment, the antagonist is an antibody to NMUR2. In another embodiment, the agent is an antibody to a ligand of NMUR2, for example, an antibody to NMU. The antibody may be polyclonal, monoclonal, chimeric, humanized, or a wholly human antibody. In another embodiment, the therapeutic method of the invention comprising administering an agent of the invention with a second pain-relieving agent, e.g., an opiate. In this embodiment, the therapeutic method may allow a decreased amount of the second agent to be administered when administered in combination with an agent of the invention.

In a fifth aspect, the invention features pharmaceutical compositions useful for treatment of pain or nociception comprising an agent capable of modulating NMUR2 activity. In one embodiment, an agent identified by a screening method of the invention.

In a sixth aspect, the invention features a method of reducing the amount of a first agent required to achieve a desired level of analgesia, by administering with the first agent, a second agent which is capable of inhibiting NMUR2. In one embodiment, the first agent is an opiate, such as morphine, and the second agent is a compound identified by the assay method of the invention or an inhibitory antibody.

In a seventh aspect, the invention features a transgenic animal comprising a modification of an endogenous NMUR2 gene. As described more fully in co-pending USSN 09/732,234 filed 07 Dec. 2000, the transgenic animal of the invention is generated by targeting the endogenous NMUR2 gene with a large targeting vector (LTVEC). In one embodiment of the transgenic animal of the invention, the animal is a knock-out wherein the NMUR2 gene is altered or deleted such that the function of the endogenous NMUR2 protein is reduced or ablated. In another embodiment, the transgenic animal is a knock-in animal modified to comprise an exogenous gene. In a more specific embodiment of the knock-in transgenic animal of the invention, the transgene is a human NMUR2 gene. Such transgenic animals are useful, for example, in identifing agents specifically inhibiting pain or sensation mediated by the human NMUR2 protein.

Other objects and advantages will become apparent from a review of the ensuing detailed description.

### BRIEF DESCRIPTION OF THE FIGURES

FIG. 1 is a bar graph showing the results of the tail flick assay for wild-type (n=10), heterozygous (n=9), and NMUR2 knock-out (n=11) mice.

FIG. 2 is a bar graph showing the results of the tail flick assay for male wild-type (n=7) and NMUR2 knock-out (n=6) mice, and female wild-type (n=3) and NMUR2 knock-out (n=5) mice.

FIG. 3 is a bar graph showing the results of the tail flick assay for wild-type (n=4 per dose) or NMUR2 knock-out (n=4 per dose) treated with 0, 2.5, 5, or 10 mg/kg of morphine.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference.

Definitions

By the term “NMUR2-associated” or “NMUR2-mediated” condition or disease is meant a condition which is affected directly or indirectly by modulation of NMUR2 activity. For example, a NMUR2-mediated condition is pain transmission.

By the term “inhibitor” is meant a substance which retards or prevents a chemical or physiological reaction or response. Common inhibitors include but are not limited to antisense molecules, antibodies, antagonists and their derivatives.

A “knock-out” animal is an animal generated from a mammalian cell which carries a genetic modification resulting from the insertion of a DNA construct targeted to a predetermined, specific chromosomal location which alters the function and/or expression of a gene that was at the site of the targeted chromosomal location. In both cases, the DNA construct may encode a reporter protein such as lacZ, protein tags, and proteins, including recombinases such as Cre and FLP. A “knock-in” animal is an animal generated from a mammalian cell which carries a genetic modification resulting from the insertion of a DNA construct targeted to a predetermined, specific chromosomal location which may or may not alter the function and/or expression of the gene at the site of the targeted chromosomal location.

General Description

This invention is based in part on elucidation of the function of the human neuromedin U receptor (NMUR2) which is a ligand for the neuropeptide neuromedin U (NMU). The experiments described below identify the function of NMU and NMUR2 as involved in the modulation of nociception, pain, and/or thermal sensation. Accordingly, these discoveries provide new methods for the treatment of NMUR2-mediated conditions, such as pain, by allowing the identification of agents capable of modulating pain transmission affected by NMUR2 activity. Further, the invention provides screening assays for identification of molecules capable of inhibiting NMUR2-mediated activity. Still further, the present invention provides methods for inhibiting NMUR2-associated activity by blocking the action of a NMUR2 ligand, including NMU.

Screening Assays

The present invention provides methods for identifying agents (e.g., candidate compounds or test compounds) that are capable of modulating (e.g., upregulating or downregulating) human neuromedin U receptor 2 (NMUR2)-mediated activity. Preferably, the invention provides methods for identifying agents capable of effecting NMUR2 modulation of nociception or pain. Agents identified through the screening method of the invention are potential therapeutics for use in providing pain relief to a subject in need thereof.

Examples of agents include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art. Test compounds further include, for example, antibodies (e.g., polyclonal, monoclonal, humanized, anti-idiotypic, chimeric, and single chain antibodies as well as Fab, F(ab′).sub.2, Fab expression library fragments, and epitope-binding fragments of antibodies). Further, agents or libraries of compounds may be presented, for example, in solution, on beads, chips, bacteria, spores, plasmids or phage.

In one embodiment, agents that bind NMUR2 are identified in a cell-based assay system. In accordance with this embodiment, cells expressing a NMUR2 protein or protein fragment are contacted with a candidate (or a control compound), and the ability of the candidate compound to bind NMUR2 is determined. The cell may be of prokaryotic origin (e.g., E. coli) or eukaryotic origin (e.g., yeast or mammalian). In specific embodiments, the cell is a NMUR2 expressing mammalian cell, such as, for example, a COS-7 cell, a 293 human embryonic kidney cell, a NIH 3T3 cell, or Chinese hamster ovary (CHO) cell. Further, the cells may express a NMUR2 protein or protein fragment endogenously or be genetically engineered to express a NMUR2 protein or protein fragment. In some embodiments of the binding assays of the invention, the compound to be tested may be labeled. Cells expressing the NMUR2 receptor are then incubated with labeled test compounds, in binding buffer, in cell culture dishes. To determine non-specific binding, unlabeled ligand may be added to the wells. After the incubation, bound and free ligands are separated and detection activity measured in each well.

The ability of the candidate compound to alter the activity of NMUR2 can be determined by methods known to those of skill in the art, for example, by flow cytometry, a scintillation assay, immunoprecipitation or western blot analysis. For example, modulators of NMUR2 activity may be identified using a biological readout in cells expressing a NMUR2 protein or protein fragment. Agonists or antagonists are identified by incubating cells or cell fragments expressing NMUR2 with test compound and measuring a biological response in these cells and in parallel cells or cell fragments not expressing NMUR2. An increased biological response in the cells or cell fragments expressing NMUR2 compared to the parallel cells or cell fragments indicates the presence of an agonist in the test sample, whereas a decreased biological response indicates an antagonist.

In more specific embodiments, detection of binding and/or modulation of a test agent to a NMUR2 protein may be accomplished by detecting a biological response, such as, for example, measuring Ca²⁺ ion flux, cAMP, IP₃, PIP₃ and transcription of reporter genes. For example, to identify ligands of NMUR2, cells expressing the receptor may be screened against a panel of know compounds utilizing a bioluminescent signal such as the aequorin luminescence assays (see, for example, Button et al. (1993) Cell. Calcium 14:663-671; Liu et al. (1999) Biochem. Biophys. Res. Comm. 266:174-178; Ungrin et al. (1999) Anal. Biochem. 272:34-42; Fujii et al. (2000) J. Biol. Chem 275:21086-21074; Raddatz et al. (2000) J. Biol. Chem. 275:32452-32459; and Shan et al. (2000) J. Biol. Chem. 275:39482-39486, which references are herein specifically incorporated by reference in their entireties). Suitable reporter genes include endogenous genes as well as exbgenous genes that are introduced into a cell by any of the standard methods familiar to the skilled artisan, such as transfection, electroporation, lipofection and viral infection. The invention further includes other end point assays to identify compounds that modulate (stimulate or inhibit) receptor activity, such as those associated with signal transduction.

In another embodiment, agents that modulate NMUR2-mediated activity are identified in a cell-free assay system. In accordance with this embodiment, a NMUR2 protein or protein fragment is contacted with a test (or control) compound and the ability of the test compound to bind NMUR2 is determined. Competitive binding may also be determined in the presence of an NMUR2 ligand. In vitro binding assays employ a mixture of components including a NMUR2 protein or protein fragment, which may be part of a fusion product with another peptide or polypeptide, e.g., a tag for detection or anchoring, and a sample suspected of containing a natural NMUR2 binding target. A variety of other reagents such as salts, buffers, neutral proteins, e.g., albumin, detergents, protease inhibitors, nuclease inhibitors, and antimicrobial agents, may also be included. The mixture components can be added in any order that provides for the requisite bindings and incubations may be performed at any temperature which facilitates optimal binding. The mixture is incubated under conditions whereby the NMUR2 protein binds the test compound. Incubation periods are chosen for optimal binding but are also minimized to facilitate rapid, high-throughput screening.

After incubation, the binding between the NMUR2 protein or protein fragment and the suspected binding target is detected by any convenient way. When a separation step is useful to separate bound from unbound components, separation may be effected by, for example, precipitation or immobilization, followed by washing by, e.g., membrane filtration or gel chromatography. One of the assay components may be labeled which provides for direct detection such as, for example, radioactivity, luminescence, optical or electron density, or indirect detection such as an epitope tag or an enzyme. A variety of methods may be used to detect the label depending on the nature of the label and other assay components, e.g., through optical or electron density, radiative emissions, nonradiative energy transfers, or indirectly detected with antibody conjugates.

It may be desirable to immobilize either the receptor protein, or fragment, or its target molecule to facilitate separation of complexes from uncomplexed forms of one of the proteins, as well as to accommodate automation of the assay. Techniques for immobilizing proteins on matrices can be used in the drug screening assays. In one embodiment, a fusion protein is provided which adds a domain that allows the protein to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of receptor-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a receptor-binding protein and a candidate compound are incubated in the receptor protein-presenting wells and the amount of complex trapped in the well can be quantitated. Methods for detecting such complexes, in addition to those described above for the GST-immobilized complexes, include immunodetection of complexes using antibodies reactive with the receptor protein target molecule, or which are reactive with receptor protein and compete with the target molecule, as well as enzyme-linked assays which rely on detecting an enzymatic activity associated with the target molecule.

In another embodiment, agents that modulate (i.e., upregulate or downregulate) NMUR2-mediated activity are identified in an animal model. Examples of suitable animals include, but are not limited to, mice, rats, rabbits, monkeys, guinea pigs, dogs and cats. In accordance with this embodiment, the test compound or a control compound is administered (e.g., orally, rectally or parenterally such as intraperitoneally or intravenously) to a suitable animal and the effect on the NMUR2-mediated activity is determined. More specifically, this method may be used to identify an agent capable of inhibiting nociception or pain transmission. Examples of assays useful for identifying potential therapeutic agents, e.g., agents capable of modulating NMUR2-mediated activity, include the tail flick assay described below, hot plate assays, or the capsicin test.

Antibodies to Human NMUR2 Protein and Ligands

According to the invention, a NMUR2 protein, protein fragment, derivative or variant, may be used as an immunogen to generate immunospecific antibodies. Further, the present invention includes antibodies to compounds capable of binding NMUR2, e.g., an NMUR2 ligand such as NMU. Such immunogens can be isolated by any convenient means, including the methods described above. Antibodies of the invention include, but are not limited to polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA ) or subclass of immunoglobulin molecule. The present invention provides for an antibody which specifically binds human NMUR2 and is useful to alleviate pain and modulate nociception mediated through NMUR2.

Therapeutic Methods and Combination Therapies

The invention is directed to therapeutically useful methods for treating any disease or condition which is improved, ameliorated, inhibited or prevented by modulation of NMUR2. Generally, inhibition of NMUR2 results in an analgesic effect, e.g., alleviation of pain or discomfort caused by pain. Inhibition of NMUR2 may be desirable in a number of situations, including for example, to alleviate pain associated with neuropathy, labor and childbirth, and/or injury. In numerous embodiments, an inhibitor of NMUR2 may be administered in combination with one or more additional compounds or therapies.

Methods of Administration

The invention provides methods of treatment comprising administering to a subject an effective amount of an agent of the invention. In a preferred aspect, the agent is substantially purified (e.g., substantially free from substances that limit its effect or produce undesired side-effects). The subject is preferably an animal, e.g., such as cows, pigs, horses, chickens, cats, dogs, etc., and is preferably a mammal, and most preferably human.

Various delivery systems are known and can be used to administer an agent of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, fibers, or commercial skin substitutes.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Pharmaceutical Compositions

The present invention also provides pharmaceutical compositions. Such compositions comprise a therapeutically effective amount of an active agent, and a pharmaceutically acceptable carrier. In a specific embodiment, the composition comprises a combination of an agent of the invention and a second pain-relieving agent. The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the active agent of the invention which will be effective in the treatment of a NMUR2-mediated condition can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Transgenic Animals

The invention includes a knock-out or knock-in animal having a modified endogenous NMUR2 gene. The invention contemplates a transgenic animal having an exogenous NMUR2 gene generated by introduction of any NMUR2-encoding nucleotide sequence which can be introduced as a transgene into the genome of a non-human animal. Any of the regulatory or other sequences useful in expression vectors can form part of the transgenic sequence. A tissue-specific regulatory sequence(s) can be operably linked to the transgene to direct expression of the NMUR2 protein to particular cells.

Knock-out animals containing a modified NMUR2 gene as described herein are useful to identify NMUR2 function. Methods for generating knock-out or knock-in animals by homologous recombination in ES cells are known to the art. Animals generated from ES cells by microinjection of ES cells into donor blastocytes to create a chimeric animal, which chimeric animal can be bred to produce an animal in which every cell contains the targeted modification. A transgenic animal can be produced by introducing nucleic acid into the male pronuclei of a fertilized oocyte, e.g., by microinjection, retroviral infection, and allowing the oocyte to develop in a pseudopregnant female foster animal. Further, random transgenic animals containing an exogenous NMUR2 gene, e.g., a human NMUR2 gene, may be useful in an in vivo context since various physiological factors that are present in vivo and that could effect ligand binding, NMUR2 activation, and signal transduction, may not be evident from in vitro cell-free or cell-based assays. Accordingly, it is useful to provide non-human transgenic animals to assay in vivo NMUR2 protein function, including ligand interaction, the effect of specific mutant NMUR2 proteins on NMUR2 protein function and ligand interaction, and the effect of chimeric NMUR2 proteins. It is also possible to assess the effect of null mutations, that is mutations that substantially or completely eliminate one or more NMUR2 protein functions.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Expression of Human NMUR2

Knock-out mice containing a lacZ gene insertion into the endogenous NMUR2 gene were generated as described in U.S. Pat. No. 6,596,54, herein specifically incorporated by reference in its entirety. LacZ expression in the knock-out mice was analyzed. The areas of the spinal cord where NMUR2 expression is highest correspond to regions of the spinal cord and medulla where unmyelinated (C-fibers) and small diameter myelinated (Aδ fibers) primary afferents terminate. These small diameter sensory afferents are known to transmit the sensation(s) of pain from the periphery to the central nervous system. More specifically, NMUR2 is expressed predominantly by neurons within the gray matter of the spinal cord and that are most abundant in the superficial layers of dorsal horn, particularly the marginal zone and substantia gelatinosa (Rexed's laminae 1 and 2) with fewer cells present in the nucleus proprius (lamina III/IV) and around the central canal (lamina X). Cells expressing NMUR2 are present in these areas throughout the length of the spinal cord, as well as in the medulla within the contiguous, homologous portions of the spinal nucleus of the trigeminal nerve. The topographic distribution of NMUR2 expressing cells in the spinal cord and medulla corresponds closely to the distribution of mu opioid receptors. The mu subclass of opioid receptors is known to be specifically involved in mediating the analgesic effects of morphine and related opiates, as well as that of endogenous opioid-like peptides (Sora et al. (1997) Proc. Natl. Acad. Sci. 94:1544-1549).

### Example 2

### Tail Flick Test

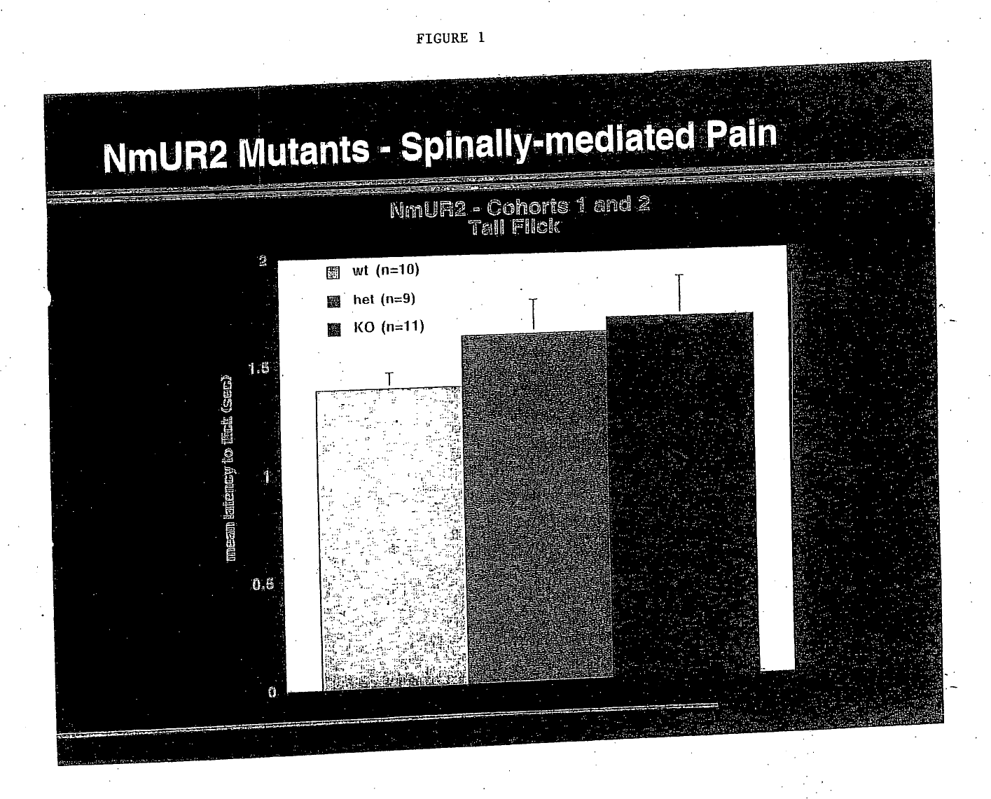

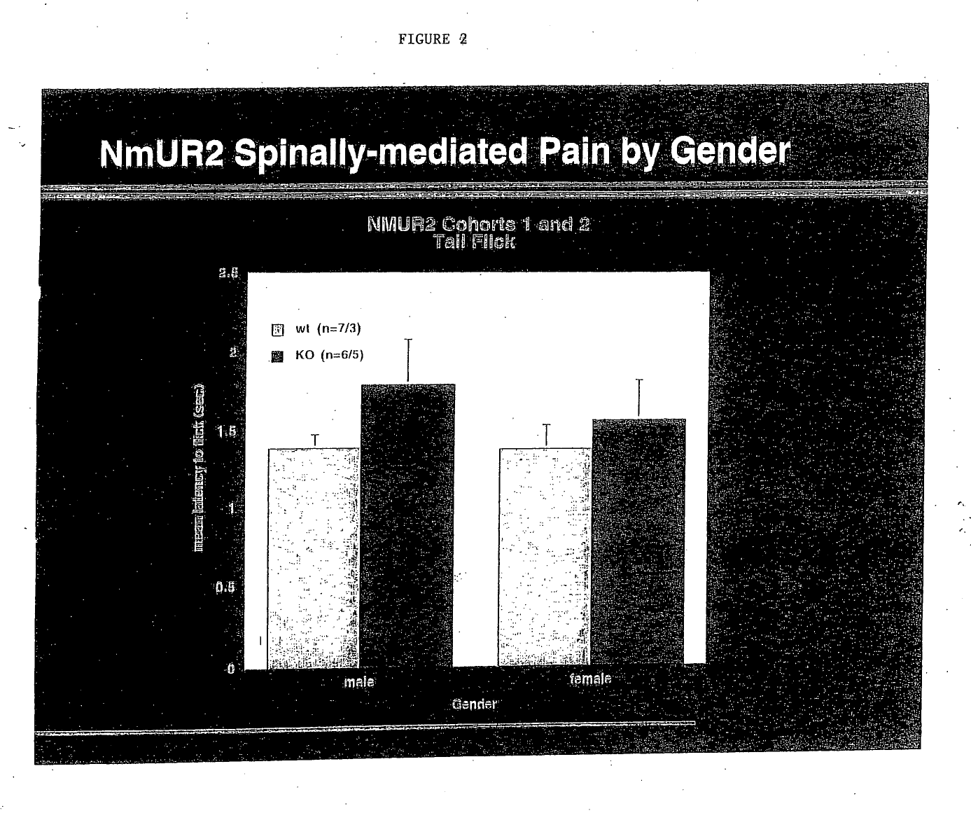

Mice were gently held on a platform of an automated apparatus wrapped in a soft cloth. Their tails were exposed and extended in a straight line along a narrow groove. Once the tail was laying flat in the groove, the experimenter activated a high-intensity and heat producing narrow beam of light that was directed at a small spot in the tail. When the animal reached its pain threshold, a spinal reflex caused the tail to “flick” out of the light beam, automatically stopping a timer that started when the beam was activated. Each animal was tested 3 times, on different regions of the tail, and the median latency to “flick” was recorded as the nociceptive threshold. Experiments were performed blind with respect to the animals' genotype. The results are shown in FIGS. 1 and 2 in wild-type, heterozygous, and NMUR2 knock-out mice (FIG. 1) and for male and female mice (FIG. 2).

### Example 3

### Effect of Morphine on NMUR2 Knock-Out Mice

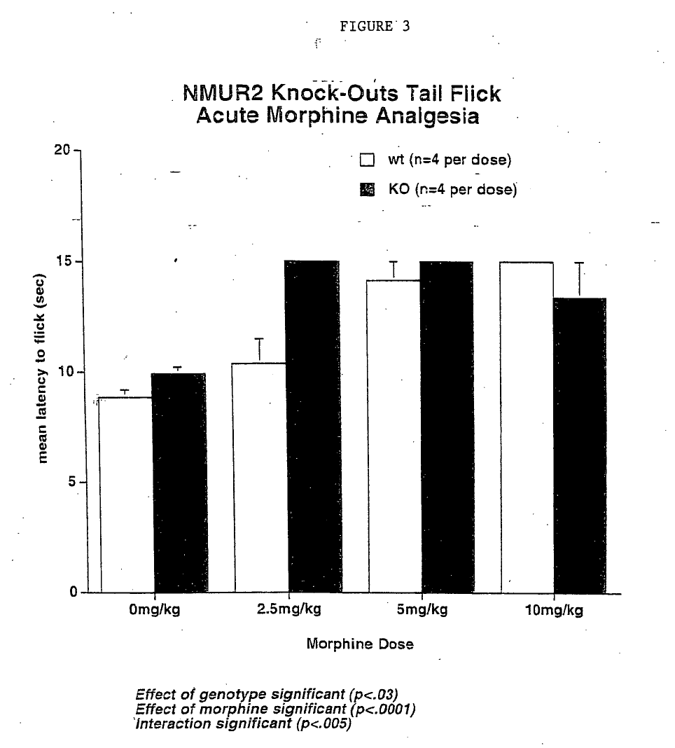

Adult male mice were treated with the mu opiate receptor agonist morphine at either 0, 2.5, 5, or 10 mg/kg sub-cutaneously. At each dose, 4 wild type mice and 4 knock-outs were treated. Tail flick latencies were determined 30 minutes after injection of morphine or vehicle, with a maximum flick latency of 15 seconds allowed to prevent tissue damage. Although all animals, regardless of genotype, showed maximal analgesia at the 5 and 10 mg/kg doses, there was a significant difference between wild types and knock-outs at the 2.5 mg/kg dose (see FIG. 3). Specifically, wild type mice showed a mild analgesia at this lowest dose of morphine tested, but NMUR2 knock-outs showed augmented analgesia, such that the mice reached the maximal 15 seconds under the light without flicking their tails. The difference between the tail flick latencies of the two genotypes at 2.5 mg/kg morphine could not be accounted for by the baseline difference in tail flick latencies normally observed between the wild types and the knock-outs, both because the difference in latency was larger, and because the enhancement of morphine analgesia is significant while the mild baseline analgesia observed in the NmUR2 knock-outs was not.

1-13. (canceled)

14. A transgenic non-human animal, comprising a modification of an endogenous NMUR2 gene.

15. The transgenic animal of claim 14, wherein the modification is an alteration or deletion of the endogenous NMUR2 gene such that the function of the endogenous NMUR2 protein is reduced or ablated.

16. The transgenic animal of claim 15, further comprising a human NMUR2 gene.
